(19) United States
(12) Patent Application Publication
(10) Pub. No.: US 2018/0071199 A1
(43) Pub. Date: Mar. 15, 2018

(54) POLYMER TREATMENT AGENT

(71) Applicant: NanoCarrier Co., Ltd., Kashiwa-shi (JP)

(72) Inventors: Kenta ISHII, Kashiwa-shi (JP); Miho KOKUBO, Kashiwa-shi (JP); Ichiro NAKATOMI, Kashiwa-shi (JP); Yasuki KATO, Kashiwa-shi (JP); Sakiko KATO, Kashiwa-shi (JP)

(21) Appl. No.: 15/553,332

(22) PCT Filed: Feb. 29, 2016
(86) PCT No.: PCT/JP2016/056105

§ 371 Date: Aug. 24, 2017

(30) Foreign Application Priority Data
Feb. 27, 2015 (JP) 2015039269

(51) Int. Cl. A61K 8/88; A61Q 5/00; A61K 8/06

(52) U.S. Cl. CPC A61K 8/88; A61K 8/06; A61Q 5/002

(57) ABSTRACT

A polymer treatment agent for rehabilitating damaged hair contains a block copolymer including a hydrophilic polymer chain segment and a hydrophobic polymer chain segment that is derived from a polyamino acid. The polymer treatment agent is not limited to methods for rehabilitating damaged head hair, but also can be applied to body hair including eyelashes and eyebrows; furthermore, it can increase the physical strength of hair even when applied in a very small amount and without using a silicone component.

### TECHNICAL FIELD

The present invention relates to a polymer treatment agent for hair, which is a novel application of a block copolymer that is a material for polymeric micelles. With regard to block copolymers that have been used as a material for polymeric micelles for drug delivery, a novel use is provided that goes beyond preconceived notions concerning their technical applicability.

### BACKGROUND ART

In an aqueous fluid (e.g., blood), block copolymers having a hydrophilic polymer chain segment derived from poly(ethylene glycol) and a hydrophobic polymer chain segment derived from poly(amino acid) form a polymeric micelle structure having a hydrophobic region in the inner shell portion caused by hydrophobic interactions between the polymers. Polymeric micelle technologies, which utilize a micelle forming mechanism caused by the hydrophobic interactions, have been studied as a technique that enables intravenous administration by solubilizing a poorly water-soluble, anti-cancer drug by holding the poorly water-soluble drug in a micelle in a sustained-releasable state, and at the same time increases drug retention in blood (Patent Literature 1). In addition, such a polymeric micelle technology has been applied to a transdermal cosmetic composition containing hinokitiol, which is a poorly water-soluble drug and a skin whitening compound; the transdermal cosmetic composition can increase the effective utilization of the whitening effect due to the long-term retention of the drug in the stratum corneum of the skin (Patent Literature 2).

### CITATION LIST

### Patent Literature

Patent Literature 1: Japanese Patent No. 2777530

Patent Literature 2: WO2008/026776

### SUMMARY OF THE INVENTION

### Problem(s) to be Solved by the Invention

It is one object of the present invention to provide, with regard to block copolymers that have been used as a material for polymeric micelles for drug delivery, a novel use that goes beyond preconceived notions concerning their technical applicability.

### Means for Solving the Problem(s)

Polymeric micelle technology is a technology aimed at sustainably delivering encapsulated compounds to a target (biological tissue) by increasing retention in pharmaceutical applications as well as in non-pharmaceutical applications such as cosmetics. In the field of polymeric micelles, usage in an environment where the polymeric micelles can be actively removed immediately after administration (e.g., the skin is washed immediately after applying the polymeric micelle solution) has been believed to significantly reduce its technical value. Thus, in the field of polymeric micelles, there is a preconceived notion that usage in an “unstable environment”, where the polymeric micelles may be physically removed immediately after use, should be avoided, and they are restricted, in principle, to usage in stable environments to exhibit their technical value.

With regard to block copolymers that have been used as a material for polymeric micelles, the present inventors found that, if a block copolymer or polymeric micelle is intentionally used as a component of a treatment agent for hair, which can be used in an “unstable environment” where it may be physically removed, the physical strength of the hair can be significantly improved and this strength improving effect can be maintained even after a washing operation, which is one example of an unstable environment, and the present invention was completed.

The present invention provides a polymer treatment agent for hair comprising a block copolymer having a hydrophilic polymer chain segment and a hydrophobic polymer chain segment.

### Effects of the Invention

According to the present invention, a polymer treatment agent for hair, which can improve the physical strength of hair even if used in only a small amount and also without using a silicone component, can be provided.

### MODES FOR CARRYING OUT THE INVENTION

The polymer treatment agent for hair contains a block copolymer having a hydrophilic polymer chain segment and a hydrophobic polymer chain segment. In the block copolymer, the hydrophilic polymer chain segment may be derived from poly(ethylene glycol), and the hydrophobic polymer chain segment may be derived from poly(amino acid). The pair of terminal ends of the main chains of the hydrophilic polymer chain segment and the hydrophobic polymer chain segment may be bound by a covalent bond.

The number of repeating units of the hydrophilic polymer chain segment can be set to, e.g., 20 or more, or e.g., 45 or more, and can be set to, e.g., 1,000 or less, or e.g., 700 or less, or e.g., 450 or less. The molecular mass of the hydrophilic polymer chain segment can be set to, e.g., 1,000 Da or more, or e.g., 2,000 Da or more, or e.g., 5,000 Da or more, and can be set to, e.g., 40,000 Da or less, or e.g., 30,000 Da or less, or e.g., 20,000 Da or less.

The number of repeating units of the hydrophobic polymer chain segment can be set to, e.g., 10 or more, or e.g., 20 or more, and can be set to, e.g., 200 or less, or e.g., 100 or less, or e.g., 60 or less. The molecular mass of the hydrophobic polymer chain segment can be set to, e.g., 1,000 Da or more, or e.g., 2,000 Da or more, and can be set to, e.g., 30,000 Da or less, or e.g., 16,000 Da or less, or e.g., 10,000 Da or less.

The hydrophobic polymer chain segment of the block copolymer may be in a state having, for example, alkyl group side chain amino acid(s) or aralkyl group side chain amino acid(s) in the repeating units. Examples of the alkyl group side chain amino acid include alanine, valine, leucine, and isoleucine. An example of the aralkyl group side chain amino acid includes phenylalanine. If it contains two or more residues of alkyl group side chain amino acids and/or aralkyl group side chain amino acids, they may be the same amino acid residues, or residues of two or more different types of alkyl group side chain amino acids and/or aralkyl group side chain amino acids may be mixed. The proportion of the residues of the alkyl group side chain amino acids or the aralkyl group side chain amino acids with respect to all of the repeating units of the hydrophobic polymer chain segment is not limited and may be, e.g., 20% or more, or e.g., 35% or more, or e.g., 40% or more, or e.g., 50% or more, or e.g., 80% or more, or e.g., 95% or more, or e.g., 99% or more, or e.g., 100%.

The molecular mass of the hydrophobic polymer chain segment with respect to the molecular mass 100% of the hydrophilic polymer chain segment can be set to, e.g., 10% or more, or e.g., 20% or more, and be set to, e.g., 400% or less, or e.g., 300% or less.

As examples of the structural formula of the block copolymer, Formulae (I) and (II) are mentioned:

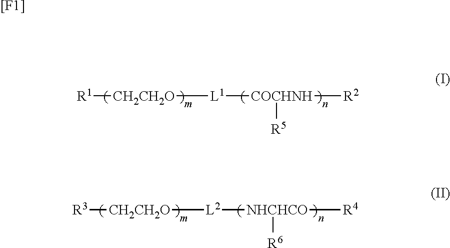

In Formulae (I) and (II), R¹ and R³ are each independently a hydrogen atom or a C₁₋₆ alkoxy, acryloxy, aryl C₁₋₃ oxy, cyano, carboxyl, amino, C₁₋₆ alkoxycarbonyl, C₂₋₇ acylamido, tri-C₁₋₆ alkylsiloxy, siloxy, or silylamino group; R² is a hydrogen atom, a saturated or unsaturated C₁-C₂₉ aliphatic carbonyl group, or an aryl carbonyl group; and R⁴ is a hydroxyl group, a saturated or unsaturated C₁-C₃₀ aliphatic oxy group, or an aryl-lower-alkyloxy group.

In Formulae (I) and (II), R⁵ and R⁶ are each independently a side chain of an amino acid. However, 50% or more, or e.g., 80% or more, or e.g., 95% or more, or e.g., 99% or more, or e.g., 100% of the n number of repeating units are a C₁-C₈ alkyl group side chain amino acid or an aralkyl group side chain amino acid. Amino acid side chains from among R⁵ or R⁶, which are not a C₁-C₈ alkyl side chain or aralkyl side chain, may be a hydrophilic moiety having an OH group or a COOH group.

In Formulae (I) and (II), m is an integer of, e.g., 20 or more, or e.g., 45 or more, or is an integer of, e.g., 700 or less, or e.g., 450 or less. n is an integer of, e.g., 10 or more, or e.g., 20 or more, or is an integer of, e.g., 200 or less, or e.g., 100 or less, or e.g., 60 or less.

In Formulae (I) and (II), L¹ is a linking group selected from —NH—, —Z—NH—, —Z—, and —Z—S—Z—NH— (where Z is independently a C₁-C₆ alkylene group); and L² is a linking group selected from —Z—, —CO—Z—CO—, —Z—CO—Z—CO—, —NH—CO—Z—CO—, and —Z—NH—CO—Z—CO— (where Z is independently a C₁-C₆ alkylene group).

As other examples of the structural formula of the block copolymer, Formulae (III) and (IV) are mentioned:

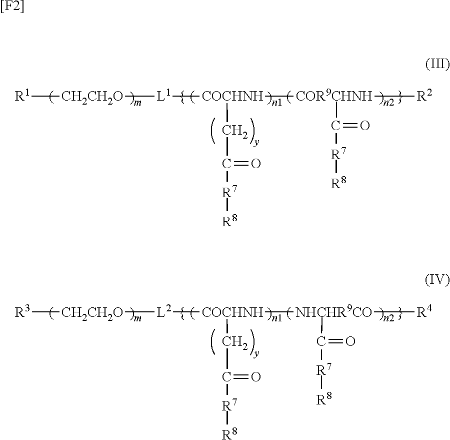

In Formulae (III) and (IV), the definitions of R¹, R², R³, R⁴, m, L¹, and L² are the same as the definitions in Formulae (I) and (II).

In Formulae (III) and (IV), R⁷ is —O— or —NH—; R⁸ is a hydrogen atom, a phenyl, benzyl, —(CH₂)₄-phenyl, or unsubstituted or amino- or carbonyl-substituted C₄-C₁₆ alkyl group, or a residue of a sterol derivative; and R⁹ is a methylene group.

In Formulae (III) and (IV), n1 is an integer of 10 to 200; n2 is an integer of 0 to 200 (however, if n2 is 1 or more, the (COCHNH) units and the (COR⁹CHNH) unit(s) are present randomly; if n2 is 2 or more, R⁸s are independently and randomly selected in each amino acid unit in the block copolymer; and hydrogen atoms account for 75% or less of all the R⁸s); and y is 1 or 2.

As other examples of the structural formula of the block copolymer, Formulae (V) and (VI) are mentioned:

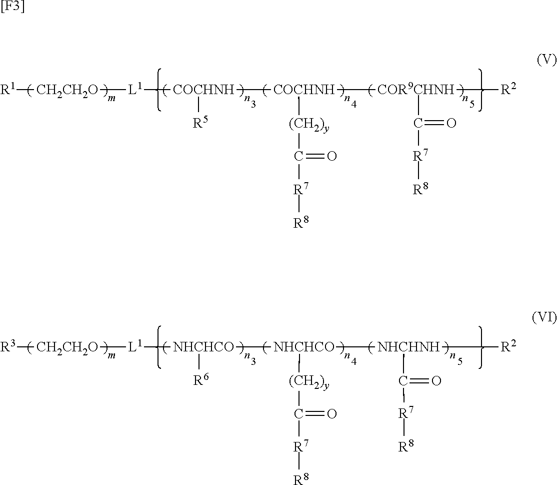

In Formulae (V) and (VI), the definitions of R⁴, R², R³, R⁴, R⁵, R⁶, L¹, and L² are the same as the definitions in Formulae (I) and (II), and the definitions of R⁷, R⁸, R⁹, and y are the same as the definitions in Formulae (III) and (IV).

In Formulae (V) and (VI), n3 is an integer of 1 to 200; n4 is an integer of 1 to 200; and n5 is an integer of 0 to 200. However, the n4 unit(s) and the n5 unit(s) (if n5 is 1 or more) are present randomly. The n3 unit(s), the n4 unit(s), and the n5 unit(s) (if n5 is 1 or more) may be present randomly, or may be present divided into a block composed of the n3 unit(s) and a block composed of the n4 unit(s) and the n5 unit(s) (if n5 is 1 or more). In addition, 50% or more, e.g., 80% or more, or e.g., 90% or more, or e.g., 95% or more, or e.g., 99% or more, or e.g., 100% from among the n3 repeating units are C₁-C₈ alkyl group side chain amino acids or aralkyl group side chain amino acids. Amino acid side chains, which are not a C₁-C₈ alkyl side chain or an aralkyl side chain, from among the n3 repeating units, may be a hydrophilic moiety having an OH group or a COOH group. In addition, the ratio of the number of the n3 unit(s) with respect to the total number of the n3 unit(s), the n4 unit(s), and the n5 unit(s) (if n5 is 1 or more) may be, e.g., 20% or more, or e.g., 35% or more, or e.g., 40% or more, or e.g., 50% or more, or e.g., 80% or more, or e.g., 90% or more.

The block copolymer can be formed by coupling, according to a known method, e.g., a polymer having a hydrophilic polymer chain and a polymer having a poly(amino acid) chain, either as is, or after purifying to narrow the molecular mass distribution if necessary. The block copolymer of Formula (I) can be prepared by, for example, forming a poly(ethylene glycol) chain by performing anionic living polymerization using an initiator capable of providing R¹, introducing an amino group at the propagating terminal end of the polymer chain, and polymerizing the desired amino acid, which contains an alkyl side chain, from the amino group terminal.

The polymer treatment agent contains one or more block copolymers described above in any combination and proportion. The polymer treatment agent may further contain a solvent. The type of solvent is not limited, and it may be an aqueous medium or a non-aqueous (oil) medium. Examples of the aqueous medium include water, and mixtures of water and a small amount of one or more water-soluble organic solvents (e.g., alcohols, such as methanol and ethanol; ketones, such as acetone; ethers, such as tetrahydrofuran and diethyl ether; DMF; and DMSO). Examples of the non-aqueous (oil) medium include non-aqueous (oil) organic solvents (e.g., liquid paraffin and ethyl oleate). The solvent may contain an additive or additives, such as a buffer, a preservative, an ultraviolet absorber, a chelating agent, an antioxidant, a redox agent, a pH adjuster, an anticoagulant, etc.

The form of the block copolymers present in the polymer treatment agent is not limited; although they may be in a non-micellar state, at least some of or substantially all the molecules may be organized and constitute polymeric micelles.

If the block copolymers assume a non-micellar state, the molecules of the block copolymers are separated from one another in the aqueous medium or in the non-aqueous (oil) medium serving as the solvent.

On the other hand, if the block copolymers constitute polymeric micelles, the polymeric micelles may be in oil-in-water micelles or water-in-oil micelles as examples thereof. In the case of oil-in-water micelles, the solvent is usually an aqueous medium, and at least some molecules of the block copolymer are radially oriented in the aqueous medium such that hydrophilic polymer chain segments are disposed outward and hydrophobic polymer chain segments are disposed inward. On the other hand, in the case of water-in-oil micelles, the solvent is usually a non-aqueous (oil) medium, and at least some molecules of the block copolymer are radially oriented in the non-aqueous (oil) medium such that hydrophilic polymer chain segments are disposed inward and hydrophobic polymer chain segments are disposed outward.

It is noted that block copolymer molecules separated from one another may coexist with block copolymer molecules forming polymeric micelles in the solvent. It is noted that, if an aqueous medium is used as the solvent, although the block copolymer molecules assume the form of oil-in-water micelles, the proportion of molecules in the non-micellar state separated from one another increases when the concentration of block copolymers is low. On the other hand, if a non-aqueous medium is used as the solvent, the block copolymer molecules usually assume the non-micellar state separated from one another. However, after adding the block copolymer molecules to the non-aqueous medium, water-in-oil micelles can be formed for at least some of block copolymer molecules by a method such as pressurizing and dispersing using a high-pressure homogenizer while adding water dropwise.

Although the reason why the polymer treatment agent of the present invention exhibits superior hair-care effects, such as increasing the physical strength of hair, is not certain, it is conjectured as follows. When the polymer treatment agent is applied to hair, the block copolymers contained in the agent enter into damage holes present in the hair in a non-micellar state or in a polymeric micelle state, and permeate into the hair. When the block copolymers enter into damage holes in the non-micellar state, oil-in-water micelles will form in a self-association manner due to residual water in the hair shaft (in the damage holes, etc.). In the case of water-in-oil type micelles, although it depends on the amount of residual water in the hair shaft (in the damage holes, etc.), if the moisture content is high, after dissociating into the non-micellar state, the structure changes into oil-in-water micelles; if the moisture content is low, the water-in-oil type structure is maintained. Thus, oil-in-water type or water-in-oil type polymeric micelles accumulate in voids in the interior of the hair. Since the interior of the hair has no polymer removal capability, the accumulation of polymeric micelle compounds composed of the block copolymers proceeds in the voids in the interior of the hair. Furthermore, when the micelle compounds densely accumulate, the cracks, into which water from the outside enters into the interior of the hair, are closed because association tends to occur, in the case of oil-in-water micelles due to interactions between hydrophilic polymer chain segments of the block copolymer, and in the case of water-in-oil micelles due to the interactions between hydrophobic segments of the block copolymer. Thus, when the voids in the interior of the hair are filled with micelle compounds, rinsing away of the micelle compounds by a washing treatment is inhibited, because the interactions between the micelle compounds are stronger than the affinity to water that is present outside of the hair; i.e., expulsion of the micelle compounds from the voids is inhibited. Thus, it is conjectured that the physical strength of the hair is significantly improved by the micelle compounds remaining in the voids in the interior of the hair due to the physical force that the micelle compounds possess. It is thus conjectured that the polymer treatment agent of the present invention functions by penetrating and remaining in the voids of the hair shaft (in particular, in the damage holes), unlike conventional transdermal treatment agents, which act on skin pores or hair roots, and conventional silicone-containing treatment agents, which are aimed at increasing the physical strength by coating the hair surface. Hence, it is conjectured that it is possible to fundamentally increase the physical strength of the hair even if it is subjected to an “unstable environment” such as washing of the hair.

It is noted that damage holes are present not only in damaged hair, but also in less damaged or substantially normal hair; in other words, they are present even in hair that is less damaged to the extent it can be treated as virgin hair. As used here in the present specification, hair, in which the percentage of the area of notches made by damage holes in the cross section of the hair shaft is 10% or more of the original area defined by the original outer circumference, is called “damaged hair”; hair, in which said area percentage is less than 10%, is called “virgin hair”. Based on the mechanism of action of the polymer treatment agent of the present invention, the effect of improving the physical strength according to the present invention is more remarkable in damaged hair. Thus, the polymer treatment agent of the present invention is more suitably applied as a damaged hair rehabilitation agent.

Although the content of the block copolymer in the polymer treatment agent is not limited, it can exhibit the effect of improving the physical strength of hair even if the block copolymer content is, e.g., 1 mass % or less, or e.g., 0.1 mass % or less, or e.g., 0.005 mass % or less, or e.g., 5×10⁻⁴ mass % or less, or e.g., 5×10⁻⁵ mass % or less, or e.g., 5×10⁻⁶ mass % or less. From the viewpoint of exhibiting the strength improving effect more reliably, the content of the block copolymer in the polymer treatment agent is, e.g., more than 0 mass %, or e.g., 0.0005 mass % or more, or e.g., 0.001 mass % or more.

The polymer treatment agent may contain additional component(s) besides the aforementioned block copolymer (and a solvent of an optional composition). Examples of the additional component(s) include, but are not limited to, a nutritional component, a preservative, an ultraviolet absorber, a chelating agent, an antioxidant, a redox agent, a pH adjuster, an anticoagulant, etc. These components may be used alone or in any combination(s) and proportion(s).

The polymer treatment agent may contain a nutritional component. Conventional hair treatment agents are intended to improve the physical strength of hair by coating the hair surface with, for example, a silicone component. Because such conventional techniques form a mere coating on the hair shaft, an apparent hair quality improving effect can be obtained, but a fundamental improvement is not possible. Furthermore, there is a dilemma in that, regardless of how much components, which are expected to have a hair quality improvement effect, or moisturizing agents, etc., are added to the treatment agent for hair, the more they are intended to improve the coating ability, the more the penetration of such a nutritional component into the interior of the hair is inhibited due to the coating on the hair surface. In contrast, because the polymer treatment agent of the present invention exhibits the effect of improving the physical strength of hair while making a silicone component unnecessarily, a fundamental improvement in hair quality can be facilitated by the nutritional component. As was described above, the polymer treatment agent of the present invention may be in a state of substantially not containing a silicone component; in principle, it is possible to infiltrate the nutritional component into the interior of hair by encapsulating the nutritional component in polymeric micelles of the block copolymer. In the present specification, the state that it substantially does not contain a silicone component means a state in which the content of a silicone component in the polymer treatment agent is in the range of less than 1 mass %, more strictly less than 0.1 mass %. On the other hand, with regard to the content of the block copolymer in the present specification, it is treated as a substantially contained state, even when it is in the range of substantially not containing a silicone component.

Although components that act on the scalp or hair roots are not positively excluded as the nutritional component, when the mechanism of action of the polymer treatment agent of the present invention is considered, the selection of a known hair quality improving component that primarily acts on the hair shaft is preferable. Examples of the hair quality improving component include keratin, ceramide, cholesterol, hematin, dilauroyl glutamic acid lysine Na, hyaluronic acid, silk protein, Erucalactone, and amino acids (glycine, alanine, valine, leucine, isoleucine, phenylalanine, serine, threonine, tyrosine, aspartic acid, glutamic acid, arginine, lysine, histidine, tryptophan, cystine, and methionine). Two or more types of nutritional components may be selected.

Although the nutritional component(s) may be contained in the polymer treatment agent in the state that it is (they are) independent from the block copolymer, it (they) may be in the state that it is (they are) encapsulated in polymeric micelles formed by the block copolymers.

The manufacturing method of the polymer treatment agent of the present invention is not limited. For example, in embodiments in which the molecules of the block copolymer are present in a non-micellar state in a solvent, the block copolymer (and other components that are optionally used) may be mixed with the solvent. On the other hand, in embodiments in which at least some molecules of the block copolymer form polymeric micelles, an appropriate manufacturing method may be selected in accordance with whether they are oil-in-water micelles or water-in-oil micelles.

For example, in the case of oil-in-water micelles, they can be formed by: i) preparing a stock solution, in which the block copolymer has been added to an organic solvent; ii) removing the organic solvent from the stock solution; iii) adding the residue (e.g., solid or paste) to water and preparing a suspension containing the block copolymer; and iv) dispersing the block copolymer in the suspension. Examples of the organic solvent used in the stock solution include acetone, dichloromethane, dimethylformamide, dimethyl sulfoxide, acetonitrile, tetrahydrofuran, and methanol. The stock solution may contain two or more types of organic solvents and may also contain a small amount of water. The organic solvent(s) may be removed from the stock solution by a known technique, such as evaporation, extraction, or membrane separation. The water, in which the residue obtained after removal of the organic solvent(s) is added, may contain an additive, such as a salt or a stabilizer. With regard to the dispersion of the mixture, known micronizing means may be used, such as a sonicator, a high-pressure emulsifying machine, or an extruder.

On the other hand, in the case of water-in-oil micelles, they can be prepared by pressurizing and dispersing with dropwise addition of water after adding the block copolymer to a non-aqueous medium as described above. Examples of the non-aqueous medium to which the block copolymer is added include non-aqueous organic solvents, such as acetone, dichloromethane, dimethylformamide, dimethyl sulfoxide, acetonitrile, and tetrahydrofuran. Two or more types of non-aqueous organic solvents may be used in combination, and it may further contain a small amount of water. The water to be added may contain an additive or additives, such as a salt, a stabilizer, etc. In the pressurizing and dispersing of the mixture, a known means, such as a high-pressure homogenizer, may be used.

The polymer treatment agent of an embodiment, in which a nutritional component is encapsulated in the polymeric micelles of the block copolymer, can be formed by mixing the nutritional component with the polymeric micelles following the formation of the polymeric micelles, or with previously-prepared polymeric micelles. The nutritional component may be mixed with the polymeric micelles in the state of a nutritional component solution containing the nutritional component, or may be mixed by dispersing it in a solution that contains the polymeric micelles (for example, the dispersion obtained in iv) above).

The polymer treatment agent of the present invention can be used for various applications relating to hair treatments; more specifically, it can be used not only for treatment of head hair, but also for treatment of body hair, including eyelashes and eyebrows. Because the polymer treatment agent can greatly improve the physical strength of hair, in which the physical strength has been significantly reduced, it can be used, for example, as a treatment agent having a damaged hair rehabilitation function, i.e. a damaged hair rehabilitation agent. In addition, for example, because the polymer treatment agent has the above-described mechanism of action, in which voids in the interior of hair are filled with the block copolymer or with the polymeric micelles constituted thereof, it can be used as a treatment agent having the function of filling voids in the interior of hair, i.e. a hair interior void filling agent. In addition, for example, because the polymer treatment agent can maintain the strength improving effect even after washing, which is an “unstable environment”, it can be used as a hair cleanser (e.g., a shampoo) to be rinsed off or as a hair modifying agent (e.g., a rinse, a conditioner, a treatment) to be rinsed off.

Examples of the form of commercial products of the polymer treatment agent for hair include liquid and paste beauty consumer products, such as shampoos, rinses, conditioners, hair packs, hair masks, treatments, styling agents, and hair dyes. Depending on the form of the commercial product, the polymer treatment agent may further contain, besides the block copolymer(s) and the nutritional component(s), for example, hydrocarbons, such as liquid paraffin, vaseline, and squalene; ester oils, such as isopropyl myristate and isopropyl palmitate; vegetable oils, such as camellia oil, olive oil, and avocado oil; nonionic surfactants, such as polyglycerol fatty acid esters, polyoxyethylene fatty acid esters, and polyoxyethylene sorbitan monolaurate; cellulose derivatives, such as methyl cellulose and hydroxyethyl cellulose; cationized polymers, such as cationized cellulose; polypeptides; preservatives; dandruff removers; chelating agents; ultraviolet absorbers; pigments; and perfumes.

### EXAMPLES

The present invention will now be described in more detail by way of examples.

### Example 1

The block copolymer of Example 1 is poly(ethylene glycol)-poly(γ-benzyl-L-glutamate) block copolymer (hereinafter referred to as “PEG-PBLG”).

PEG-PBLG was prepared as follows. PEG-NH₂ (molecular mass: 10,000 Da) was dissolved in dehydrated dimethylformamide under an argon atmosphere; BLG-NCA, which is the α-amino acid-N-carboxy anhydride (NCA) for polymerization of the PBLG segment, was added in an amount of 42 equivalents to PEG-NH₂, and the mixture was agitated at 40° C. for 18 hours. The resultant reaction mixture was precipitated in a mixed solvent of hexane and ethyl acetate (1:1) and then washed with the same solvent. After drying, a PEG-PBLG powder was obtained. From ¹H-NMR analysis, the degree of polymerization of the PEG segment in the PEG-PBLG was 227 and the degree of polymerization of the PBLG segment in the PEG-PBLG was 40. The structural formula of PEG-PBLG is represented by Formula (1).

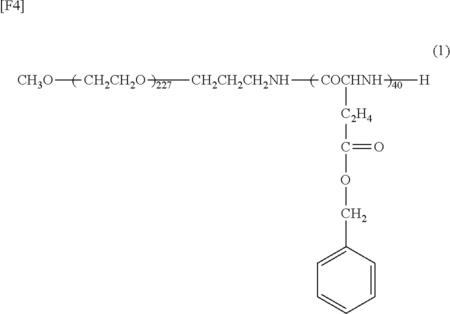

### Example 2

The block copolymer of Example 2 is poly(ethylene glycol)-polyleucine block copolymer (hereinafter referred to as “PEG-pLeu”).

PEG-pLeu was prepared in the same manner as in Example 1 except that Leu-NCA was used, in an amount of 44 equivalents, as the NCA. From ¹H-NMR analysis, the degree of polymerization of the pLeu segment was 40. The structural formula of PEG-pLeu is represented by Formula (2).

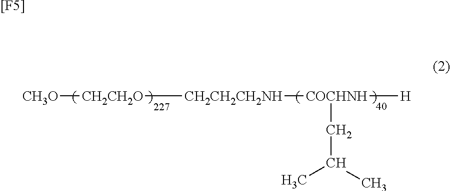

### Example 3-1

The block copolymer of Example 3-1 is a poly(ethylene glycol)-poly(leucine/γ-benzyl-L-glutamate) block copolymer composed of a PEG segment and a poly(leucine/γ-benzyl-L-glutamate) segment containing in a random manner 25 mol % leucine (Leu) units and 75 mol % γ-benzyl-L-glutamate (BLG) units. Hereinafter, the mixed-type copolymer having such Leu and BLG units will be referred to as “PEG-p(Leu/BLG)”; in case the molar ratio of these units is indicated, it will be referred to as “PEG-p(Leu/BLG) (75:25).”

PEG-p(Leu/BLG) (75:25) was prepared in the same manner as in Example 1 except that Leu-NCA and BLG-NCA were used as the NCA, and the molar ratio of these NCAs was adjusted to achieve a molar ratio of Leu units and BLG units of 75:25. From ¹H-NMR analysis, the degree of polymerization of the PEG segment in PEG-p(Leu/BLG) (75:25) was 227 and the degrees of polymerization of the Leu and BLG units in the p(Leu/BLG) segment were 30 and 10, respectively.

The structural formula of PEG-p(Leu/BLG) (25:75) is represented by Formula (3). For the sake of convenience, although the Leu and BLG units are illustrated on the left and right sides, respectively, in the curly brackets { } of Formulae (3), (4), and (5) described below, in fact, these units are randomly disposed.

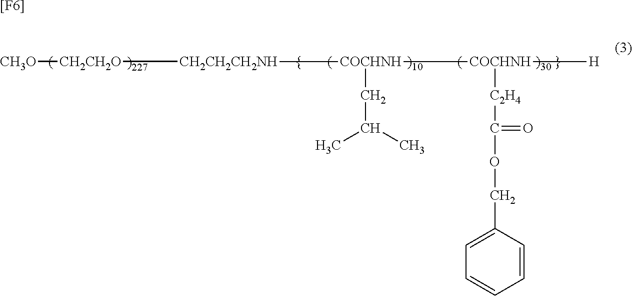

### Example 3-2

The block copolymer of Example 3-2 is PEG-p(Leu/BLG) (50:50). PEG-p(Leu/BLG) (50:50) was prepared in the same manner as in Example 3-1 except that the molar ratio of the NCAs was adjusted to achieve a molar ratio of Leu units and BLG units of 50:50. The structural formula of PEG-p(Leu/BLG) (50:50) is represented by Formula (4).

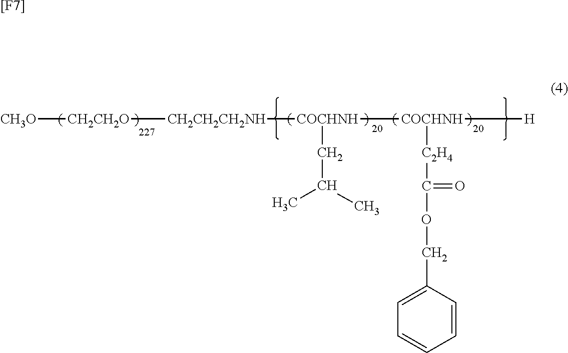

### Example 3-3

The block copolymer of Example 3-3 is PEG-p(Leu/BLG) (75:25). PEG-p(Leu/BLG) (75:25) was prepared in the same manner as in Example 3-1 except that the molar ratio of the NCAs was adjusted to achieve a molar ratio of Leu units and BLG units of 75:25. The structural formula of PEG-p(Leu/BLG) (75:25) is represented by Formula (5).

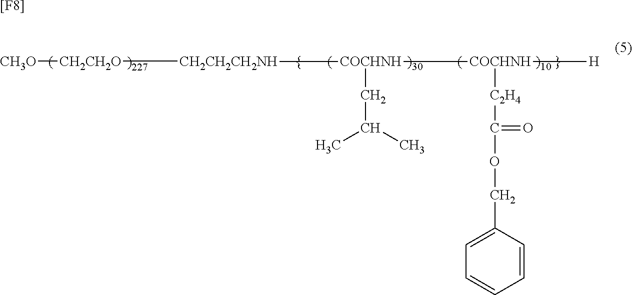

[Evaluation 1]

A human hair sample (BS-PGM, available from Beaulax) was immersed in a 0.5% aqueous SDS solution and agitated for 30 minutes, then washed with distilled water and dried with a cool air dryer. The washed and dried hair sample was divided into two groups, and one group was used as a virgin hair sample. The other hair group was used to prepare a damaged hair sample. The damaged hair sample was prepared by coating the latter hair group with a three-component bleaching agent (Gatsby EX Hi-Bleach, manufactured by Mandom Corporation) and then allowing it to stand still for 30 minutes; after immersing in a 0.5% aqueous SDS solution and agitating for 30 minutes, it was washed with distilled water and dried with a cool air dryer.

An aqueous dispersion that used water, which is an aqueous medium, as the solvent was prepared as the polymer treatment agent. The block copolymers of each of the Examples were prepared as stock solutions by adding water so that they became 10 mg/mL; after being agitated overnight, they were subjected to a Nanovater treatment (150 MPa, 10 passes). Polymer treatment agents having different block copolymer concentrations were prepared by adding water to the stock solutions. More specifically, in the polymer treatment agents, at least some molecules of the block copolymer in the aqueous dispersion serving as the solvent are in the state of polymeric micelles, in which they are radially oriented such that hydrophilic polymer chain segments are oriented outward and hydrophobic polymer chain segments are oriented inward.

Each hair sample was immersed in a polymer treatment agent for 60 minutes, and then washed with distilled water and dried with a cool air dryer.

The flexural rigidity of each hair sample was measured with a single fiber bending tester (KES-FB2-SH, manufactured by KATO TECH CO., LTD.). The measurement environment was a temperature of 20° C. and a humidity of 60%. The set conditions of the testing machine were: sense value: 1, curvature: ±2.5 cm⁻¹, measuring length: 1 cm, number of measurements: 1. The flexural rigidity was analyzed by the KES-FB2-SYSTEM data measuring program manufactured by KATO TECH CO., LTD.

In order to consider the influence of variation due to hair size and to average the measurement results of the flexural rigidity, the diameter of each strand of the hair sample was measured with a hair diameter measurement system (manufactured by KATO TECH CO., LTD.) and the cross-sectional areas of the hair samples were calculated.

The value of the flexural rigidity per unit cross-sectional area was calculated according to Formula (i) for an untreated hair sample as well as for hair samples treated with the polymer treatment agents containing the block copolymers of each of the Examples.

L=B/M (i)

In the above Formula (i), L is the flexural rigidity per unit cross-sectional area (gf·cm²/mm²) of the hair sample, B is the flexural rigidity of the hair sample (gf·cm²), and M is the cross-sectional area (mm²) of the hair sample.

The ratios of the values of the flexural rigidity of the hair samples treated with the polymer treatment agents, with the flexural rigidity of the untreated hair sample being assumed to be 1, were calculated according to Formula (i). The results are shown in Table 1.

TABLE 1

| | Block copolymer | | Flexural rigidity ratio | |
|---|---|---|---|---|
| | | Concentration | Virgin hair | Damaged hair |
| | Type | (w/w %) | sample | sample |
| Example 1 | PEG-PBLG | 1 | 3.50 | 4.85 |
| | | 0.05 | — | 3.89 |
| | | 0.005 | 1.59 | 3.30 |
| Example 2 | PEG-pLeu | 1 | 1.36 | 9.77 |

As shown in Table 1, polymer treatment agents of the present invention can increase the physical strength with respect to both a virgin hair sample and a damaged hair sample. In addition, the strength improving effect was particularly remarkably exhibited in the damaged hair samples.

[Evaluation 2]

Polymer treatment agents having different block copolymer concentrations were prepared in the same manner as in Evaluation 1 except that stock solutions were prepared by using the block copolymers of Examples 1, 2, and 3-1 to 3-3. In the polymer treatment agents, at least some molecules of the block copolymer are in the form of polymeric micelles as in the polymer treatment agents of Evaluation 1.

The flexural rigidity was measured in the same manner as in Evaluation 1 except for using a damaged hair sample, in which the stand still time after applying a bleaching agent to BS-PG (Spec: K-085) of Beaulax Co. was changed from 30 minutes to one hour.

TABLE 2

| | Block copolymer | | |
|---|---|---|---|
| | | Concentration | |
| | Type | (w/w %) | Flexural rigidity ratio |
| Example 1 | PEG-PBLG | 1 | 1.42 |
| | | 0.05 | 1.28 |
| | | 0.005 | 1.17 |
| | | 0.001 | 1.17 |
| Example 3-1 | PEG-p(Leu/BLG) | 1 | 1.45 |
| | (25:75) | 0.005 | 1.21 |
| | | 0.001 | 1.21 |
| Example 3-2 | PEG-p(Leu/BLG) | 1 | 1.49 |
| | (50:50) | 0.005 | 1.19 |
| | | 0.001 | 1.17 |
| Example 3-3 | PEG-p(Leu/BLG) | 1 | 1.35 |
| | (75:25) | 0.005 | 1.18 |
| | | 0.001 | 1.16 |
| Example 2 | PEG-pLeu | 1 | 1.65 |
| | | 0.05 | 1.41 |
| | | 0.005 | 1.24 |
| | | 0.001 | 1.23 |

As shown in Table 2, the polymer treatment agent of the present invention can improve the physical strength of hair even if the block copolymer content is very low; i.e., 0.005 mass % or less, or 0.001 mass % or less. It is noted that, although the numerical values of the flexural rigidity differ from the case of Evaluation 1 with respect to polymer treatment agents having equal contents of the block copolymers of Example 1 or 2, this is merely a difference caused by a difference of the preparation conditions of the damaged hair samples. In addition, even though the damaged hair sample is more severely damaged than in the case of Evaluation 1, the polymer treatment agent of the present invention can improve the physical strength even for such severely damaged hair.

[Evaluation 3]

A cream agent that used a cream, which is a non-aqueous medium, was prepared as the polymer treatment agent. The cream was prepared by stirring and mixing 15.3 (w/w) % liquid paraffin and 7.6 (w/w) % Tween 80 until uniform. The polymer treatment agent was prepared as the cream agent by adding 76.3 (w/w) % of the stock solution (aqueous dispersion) containing 1 (w/w) % of the block copolymer of Example 1 or 2, which was prepared in Evaluation 1, to the cream, further adding 0.8 (w/w) % Xanthan gum, which is a thickener, in several portions, and stirring gently overnight. The final concentration of the block copolymer in the polymer treatment agent is 0.8 (w/w) %. In the polymer treatment agent, at least some molecules of the block copolymer are in the state of being separated from one another in the solvent.

In addition, as a Comparative Example, a cream agent was prepared in the same manner except that the above-described stock solution was not added.

The values of the flexural rigidity of the hair samples per unit cross-sectional area was calculated in the same manner as in Evaluation 2 except that the cream agent was used as the polymer treatment agent. The ratios of the flexural rigidity of the hair samples in accordance with each polymer treatment agent, in the case that the value of the flexural rigidity is taken as 1 based on the cream agent of Comparative Example, are shown in Table 3 below.

TABLE 3

| | Block copolymer | | |
|---|---|---|---|
| | | Concentration | |
| | Type | (w/w %) | Flexural rigidity ratio |
| Example 1 | PEG-PBLG | 0.8 | 1.25 |
| Example 2 | PEG-pLeu | 0.8 | 1.45 |

As shown in Table 3, the polymer treatment agent of the present invention can improve the physical strength of hair even in a state, in which a non-aqueous medium is used as a solvent; i.e., in the state that at least some molecules of the block copolymers are separated from one another in the solvent.

### INDUSTRIAL APPLICABILITY

The polymer treatment agent of the present invention can improve the physical strength of hair; because the physical strength increasing effect can be exhibited even when a small amount is used, it is not limited to the care of head hair and it can be applied to uses including body hair, including eyelashes and eyebrows; it can be particularly suitably used in the beauty care field.

1. A method for rehabilitating damaged hair comprising:
applying a polymer treatment agent comprising a block copolymer disposed in an aqueous medium or a non-aqueous medium to the damaged hair to rehabilitate the damaged hair, the block copolymer having a hydrophilic polymer chain segment and a hydrophobic polymer chain segment that is derived from a polyamino acid.

2.-3. (canceled)

4. The method according to claim 1, wherein:
the block copolymer is disposed in the non-aqueous medium; and
at least some molecules of the block copolymer are in a state of being separated from one another in the non-aqueous medium.

5. The method according to claim 1, wherein:
the block copolymer is disposed in the aqueous medium; and
at least some molecules of the block copolymer are in the state of having formed a polymeric micelle in the aqueous medium.

6. The method according to claim 5, wherein the block copolymer is disposed in the aqueous medium, and the polymeric micelle is oriented such that hydrophilic polymer chain segments of the block copolymer are disposed radially outward and hydrophobic polymer chain segments of the block copolymer are disposed radially inward.

7. (canceled)

8. The method according to claim 1, wherein the polymer treatment agent contains more than 0 mass % to 1 mass % or less of the block copolymer.

9. The method according to claim 8, wherein the polymer treatment agent contains more than 0 mass % to 0.1 mass % or less of the block copolymer.

10. The method according to claim 9, wherein the polymer treatment agent contains more than 0 mass % to 0.005 mass % or less of the block copolymer.

11.-12. (canceled)

13. The method according to claim 1, wherein the hydrophobic polymer chain segment that is derived from a polyamino acid includes repeating units of alkyl- or aralkyl group side chain amino acid residues.

14. The method according to claim 13, wherein the molar proportion of the repeating units of the alkyl- or aralkyl group side chain amino acid residues is 20% or more relative to all amino acid residues in the hydrophobic polymer chain segment.

15.-16. (canceled)

17. The method according to claim 16, wherein said molar proportion is 50% or more.

18. The method according to claim 1, wherein the polymer treatment agent contains more than 0 mass % to 0.01 mass % or less of the block copolymer.

19. The method according to claim 1, further comprising rinsing the damaged hair after applying the polymer treatment agent to the damaged hair.

20. The method according to claim 6, wherein:
the hydrophobic polymer chain segment that is derived from a polyamino acid includes repeating units of alkyl- or aralkyl group side chain amino acid residues;
the polymer treatment agent contains more than 0 mass % to 1 mass % or less of the block copolymer; and
the method further comprises rinsing the damaged hair within 60 minutes after applying the polymer treatment agent to the damaged hair.

21. The method according to claim 4, wherein:
the block copolymer is disposed in a non-aqueous medium;
the hydrophobic polymer chain segment that is derived from a polyamino acid includes repeating units of alkyl- or aralkyl group side chain amino acid residues;
the polymer treatment agent contains more than 0 mass % to 1 mass % or less of the block copolymer; and
the method further comprises rinsing the damaged hair within 60 minutes after applying the polymer treatment agent to the damaged hair.

22. The method according to claim 1, wherein the hydrophobic polymer chain segment has a molecular mass of 1,000-30,000 Da.

23. The method according to claim 22, wherein the hydrophilic polymer chain segment has a molecular mass of 1,000-40,000 Da.

24. The method according to claim 23, wherein:
the hydrophobic polymer chain segment has a molecular mass of 2,000-16,000 Da; and
the hydrophilic polymer chain segment has a molecular mass of 2,000-20,000 Da.

25. The method according to claim 24, wherein:
the hydrophilic polymer chain segment comprises poly(ethylene glycol); and
the hydrophobic polymer chain segment contains at least 50% molar percent of amino acid residues selected from the group consisting of alanine residues, valine residues, leucine residues, isoleucine residues and phenylalanine residues.

26. The method according to claim 24, wherein:
the hydrophilic polymer chain segment comprises poly(ethylene glycol); and
the hydrophobic polymer chain segment comprises polyleucine or a mixed polymer of γ-benzyl-L-glutamate and leucine, the mixed polymer containing the γ-benzyl-L-glutamate and leucine arranged either in blocks or randomly.

27. A kit for rehabilitating damaged hair comprising:
a container containing a polymer treatment agent comprising a block copolymer disposed in an aqueous medium or a non-aqueous medium, the block copolymer having a hydrophilic polymer chain segment and a hydrophobic polymer chain segment that is derived from a polyamino acid; and
a label or product insert associated with the container and providing instructions for applying the polymer treatment agent to the damaged hair to rehabilitate the damaged hair.

28. The kit according to claim 27, wherein:
the block copolymer is disposed in the aqueous medium,
at least some molecules of the block copolymer are in the state of having formed polymeric micelles, and
the polymeric micelles are arranged such that hydrophilic polymer chain segments of the block copolymer are disposed radially outward and hydrophobic polymer chain segments of the block copolymer are disposed radially inward.

29. The kit according to claim 27, wherein the polymer treatment agent contains more than 0 mass % to 1 mass % or less of the block copolymer.

30. The kit according to claim 27, wherein the polymer treatment agent contains more than 0 mass % to 0.01 mass % or less of the block copolymer.

31. The kit according to claim 27, wherein the hydrophobic polymer chain segment that is derived from a polyamino acid includes repeating units of alkyl- or aralkyl group side chain amino acid residues.

32. The kit according to claim 31, wherein the repeating units of alkyl- or aralkyl group side chain amino acid residues constitute at least 50 molar percent of all amino acid residues in the hydrophobic polymer chain segment.

33. The kit according to claim 32, wherein the polymer treatment agent contains more than 0 mass % to 0.01 mass % or less of the block copolymer.

34. The kit according to claim 27, wherein the label or product insert provides instructions for rinsing the damaged hair within 60 minutes after applying the polymer treatment agent to the damaged hair.

35. The method according to claim 32, wherein the polymer treatment agent further comprises at least one hair quality improving compound selected from the group consisting of keratin, ceramide, cholesterol, hematin, dilauroyl glutamic acid lysine Na, hyaluronic acid, silk protein, Erucalactone, glycine, alanine, valine, leucine, isoleucine, phenylalanine, serine, threonine, tyrosine, aspartic acid, glutamic acid, arginine, lysine, histidine, tryptophan, cystine, and methionine.

36. A method for rehabilitating damaged hair comprising:
applying a polymer treatment agent comprising a block copolymer disposed in an aqueous medium or a non-aqueous medium to the damaged hair to rehabilitate the damaged hair, the block copolymer having one of the following formulae: 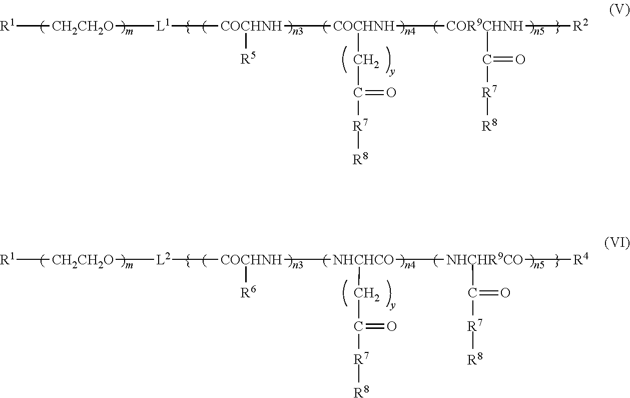
wherein:
R¹ and R³ are each independently a hydrogen atom or a C₁₋₆ alkoxy, acryloxy, aryl C₁₋₃ oxy, cyano, carboxyl, amino, C₁₋₆ alkoxycarbonyl, C₂₋₇ acylamido, tri-C₁₋₆ alkylsiloxy, siloxy, or silylamino group;
R² is a hydrogen atom, a saturated or unsaturated C₁-C₂₉ aliphatic carbonyl group, or an aryl carbonyl group;
R⁴ is a hydroxyl group, a saturated or unsaturated C₁-C₃₀ aliphatic oxy group, or an aryl-lower-alkyloxy group
R⁵ and R⁶ are each independently a side chain of an amino acid, with the proviso that 50% or more of the n3 number of R⁵ and R⁶ are a C₁-C₈ alkyl group or an aralkyl group;
R⁷ is —O— or —NH—;
R⁸ is a hydrogen atom, a phenyl, benzyl, —(CH₂)₄-phenyl, or unsubstituted or amino- or carbonyl-substituted C₄-C₁₆ alkyl group, or a residue of a sterol derivative;
R⁹ is a methylene group;
L¹ is a linking group selected from —NH—, —Z—NH—, —Z—, and —Z—S—Z—NH—, where Z is independently a C₁-C₆ alkylene group;
L² is a linking group selected from —Z—, —CO—Z—CO—, —Z—CO—Z—CO—, —NH—CO—Z—CO—, and —Z—NH—CO—Z—CO—, where Z is independently a C₁-C₆ alkylene group,
m is an integer of 20-700;
n3 is an integer of 1 to 200;
n4 is an integer of 0 to 200;
n5 is an integer of 0 to 200, with the provisos that 20≦(n3+n4+n5)≦200 and 0.5≦[n3/(n3+n4+n5)]; and
y is 1 or 2.

37. The method according to claim 36, wherein the polymer treatment agent contains more than 0 mass % to 0.01 mass % or less of the block copolymer.

38. The method according to claim 36, further comprising rinsing the damaged hair within 60 minutes after applying the polymer treatment agent to the damaged hair.

39. The method according to claim 36, wherein the polymer treatment agent is applied to the damaged hair for a sufficient time to increase flexural rigidity of the damaged hair and then the damaged hair is rinsed.
